(19) United States
(12) Patent Application Publication
(10) Pub. No.: US 2024/0024397 A1
(43) Pub. Date: Jan. 25, 2024

(54) Methods and Processes for Obtaining Extracts from Natural Ingredients

(71) Applicant: Gary Grey, New York, NY (US)

(72) Inventor: Gary Grey, New York, NY (US)

(21) Appl. No.: 17/870,495

(22) Filed: Jul. 21, 2022

(51) Int. Cl. A61K 36/185; A61K 36/738; B01D 11/02; B01D 3/14; B01D 5/00

(52) U.S. Cl. CPC A61K 36/185; A61K 36/738; B01D 11/0296; B01D 11/0219; B01D 3/143; B01D 5/006; A61K 2236/10; A61K 2236/37; B01D 2011/007

(57) ABSTRACT

The present technology is directed to methods and processes for optimally extracting and distilling desired natural ingredients from raw natural ingredients, involving pre-soaking the raw natural ingredients in a solvent, boiling the liquid component from the pre-soaking step, allowing the resultant vapor to contact the solid component, and passing the resultant enhanced vapor through a distillation column to obtain a desired product. Any steps of the methods and processes herein can be performed in conjunction with a vacuum or pressurizing device—that is, at an elevated or lowered pressure compared with ambient pressure.

### BACKGROUND

The present technology relates to methods and processes for obtaining natural ingredients and other desirable components from biological sources; in particular, methods and processes that involve extraction and distillation.

Biological sources such as plant matter are often subject to steam distillation and extraction order to obtain desirable ingredients for cosmetic and medicinal purposes. Since such biological sources often only make small amounts of desired metabolites, a great deal of the raw material is often needed to produce a useable amount of a desired end product.

In known methods and processes for obtaining desired ingredients from biological sources, plant matter is typically placed either directly in boiling water in a batch process using a still, or in a basket through which steam passes (sometimes referred to in the industry as a “gin basket”). In such methods and processes, the resulting steam is typically then reconstituted in a cooled column—producing an aqueous solution of plant matter, also known as “hydrosol” (also referred to interchangeably as “herbal distillate,” “floral water,” “hydrolate,” “herbal water” or “essential water”). Such hydrosols are mainly mixtures essential oils and water soluble components obtained by steam distillation (hydrodistillation) from biological sources. This hydrosol can either be deemed the final product, or further refined and purified to produce the final product.

However, ordinary steam distillation is often problematic because the surface area of the plant matter is limited, and plant internals are often not fully exposed. This can leave desired components behind, leading to inefficiencies and wasted material. It also requires a great amount of energy, making it costly.

Therefore, an ongoing need exists for methods and processes that can predictably and efficiently extract and purify desired natural ingredients from natural sources in a manner that maximizes the extraction, while also saving energy by avoiding the generation of excess heat.

### SUMMARY

In certain embodiments, the present technology is directed to a method for obtaining a natural ingredient from a biomass, the method comprising the steps of:
(a) pre-soaking a quantity of the biomass in a solvent, yielding a solid component and a liquid component;
(b) transferring the liquid component into a boiler, the boiler having a space through which moves a steam supply generated from the boiler moves;
(c) transferring the solid component into a botanical basket that is configured such that the steam supply generated from the boiler, when moving, passes through the botanical basket and contacts the solid component in the botanical basket, yielding an enhanced vapor stream that exits the botanical basket and enters a distillation column;
(d) passing the enhanced vapor stream through the distillation column, yielding a distillate stream,
(e) directing the distillate stream from the distillation column to a condenser maintained at a temperature lower than the temperature of the distillate stream as it exits the distillation column, allowing for the creation of a condensate from at least a portion of the distillate stream; and
(f) capturing the condensate in a collection tank;
   wherein one or more of steps (a) through (f) is performed at a pressure of 200 mmHg (0.263 atm) to 760 mmHg (1 atm).

In certain embodiments, the present technology is directed to a process encompassing any step of any method discussed herein; as well as to various compositions made with the methods and processes discussed herein.

### BRIEF DESCRIPTION OF THE DRAWINGS

FIG. 1 shows an exemplary pre-soaking step in certain embodiments of the present technology.

FIG. 2 shows a schematic of an exemplary embodiment method or process herein.

FIG. 3 shows a schematic of another exemplary embodiment method or process herein.

### DETAILED DESCRIPTION

All ingredient and formulation percentages herein are denoted in % w/w, unless explicitly noted in specific formulations. All ingredients are at 100% purity/concentration unless otherwise noted.

As used herein, “primarily” means more than 50% of a quantity or value. As used herein, “substantially” means within 5% of a quantity or value—for example, “substantially pure” means containing no more than 5% impurities.

As used herein, “essentially free of” an ingredient means containing less than 5% w/w of such ingredient. As used herein, “completely free of” an ingredient means containing none of that ingredient.

As used herein, “natural” describes any material that comes from the earth, rather than being synthesized in a lab or facility. As used herein, “biological” means any natural material that comes from a living organism. As used herein, “biomass” means any natural or biological ingredient from which an end product is desired to be extracted or separated. Biomass can comprise any natural or biological material, including organic (that is, carbon-containing) material including, but not limited to, material from a plant, animal, fungus, bacteria, any other organism, or mineral. In various embodiments herein, the biomass comprises a plant. In various embodiments herein, the biomass comprises a raw natural ingredient, such as any part of a plant—for example, leaves, twigs, roots, stems or the like.

In various embodiments, the present technology is directed to methods and processes that involve pressing, or extracting and pressing, a biomass prior to boiling and distillation. In such embodiments, the solid portion of pressed biomass can be used in the still's basket and liquid extract is used in the water. This can produce more full spectrum distillate or “hydrosol” with desired properties; it can also lead to greater efficiency in the overall process, and less waste of materials and energy.

### Pre-Soaking and Liquid Extraction

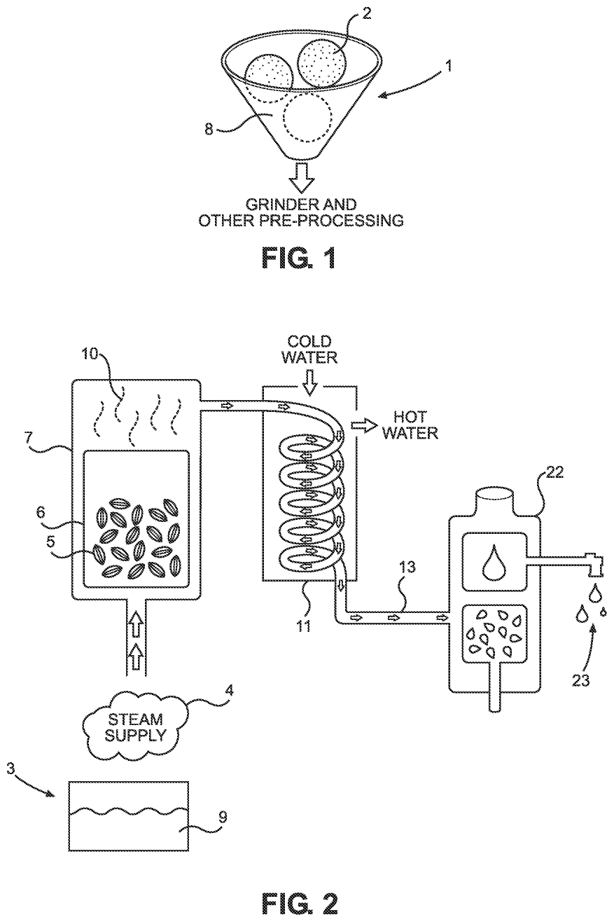

In various embodiments, methods and processes herein involve a pre-soaking step. This step can be conducted in, for example, a tank or other container in which the raw natural ingredient from which the extract is to be obtained (the biomass) is soaked in solvent. FIG. 1 illustrates an exemplary embodiment with a tank 1 in which biomass 2 is held in a batch process. In various embodiments, the tank 1 can be any shape that holds the biomass 2 and solvent 8, and can or may not have any openings other than the open top typically found in such a tank. Another embodiment of a tank 1 holding biomass 2 for pre-soaking in solvent 8 is shown in FIG. 3.

In various embodiments, the raw natural ingredient is at least partially, substantially entirely, or entirely in solid form, and at least partially, substantially entirely, or entirely submerged in the solvent. In various embodiments, the solvent comprises water, alcohol, a plant oil, or glycerin, or any other commonly used solvent known to one of ordinary skill in the art.

In various embodiments, the pre-soaking step can occur from a period of time ranging from 2 to 48 hours, or 4 to 40 hours, or 6 to 36 hours, or 2 to 24 hours, or 2 to 12 hours, or 12 to 40 hours, or 18 to 48 hours, or 18 to 24 hours. For example, for leafy plants, the pre-soaking step can occur from, e.g., 12 to 40 hours or 18 to 36 hours. For grains, the pre-soaking step can occur from, e.g., 2 to 24 hours or 6 to 18 hours. For woody materials such as roots and barks, the pre-soaking step can occur from, e.g., 18 to 48 hours or 24 to 36 hours. In various embodiments, the pre-soaking time is optimized to extract as much of the desired end product from the biomass as possible.

In various embodiments, the pre-soaking step results in a solid component and a liquid component. The solid component can comprise what remains of the biomass, and the liquid component can comprise the solvent in which the biomass was soaked, infused with any material obtained from the biomass through the contact with the solvent, including any desired end products of the methods and processes herein. In certain embodiments, the solid component and the liquid component can then be separated, for example, through a pressing step. Of course, in certain embodiments, some liquid will remain in the solid component, and some solid particulates will remain in the liquid component.

### Boiler and Botanical Basket

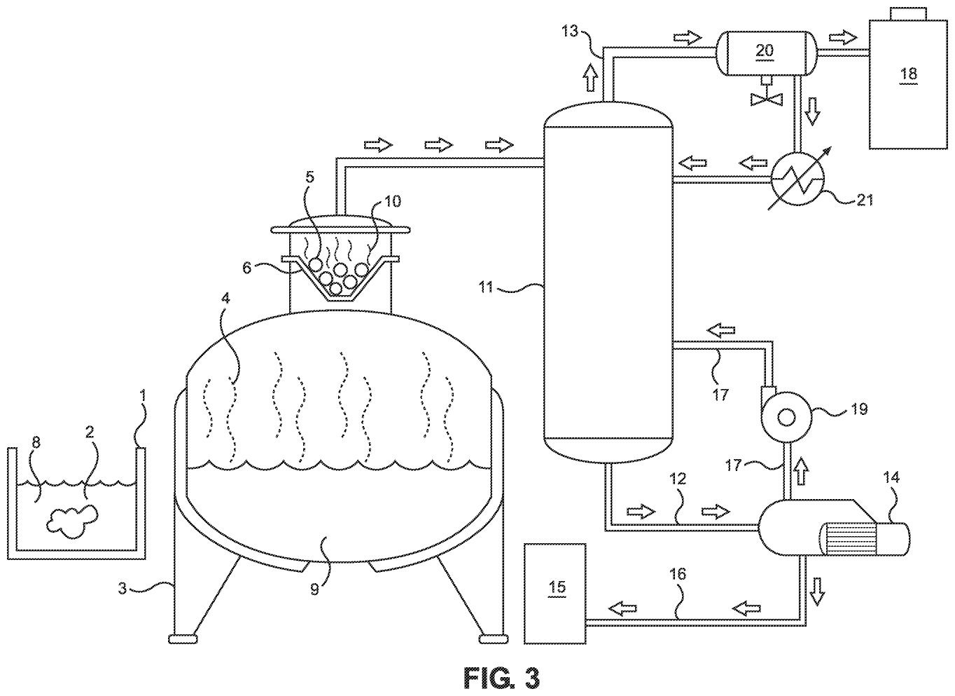

After the solid component and the liquid component are formed, in certain embodiments, the liquid component is then transferred to a boiler (also referred to herein as a “kettle”) and boiled. Exemplary embodiments are shown in FIG. 2 and FIG. 3. As can be seen for example, in FIG. 2, the liquid component 9 can be placed in the boiler 3, and heated up to boiling, generating a steam supply 4, which is then passed through the botanical basket 6. The solid component 5 can be placed within the botanical basket 6, such that the steam supply 4 flows from the boiler and contacts the solid component 5.

As discussed above, “botanical basket” refers herein to a structure that is sometimes referred to in the industry as a “gin basket.” In various embodiments, the botanical basket can be located before, inside, or after the column, as will be discussed in further detail later. In certain embodiments, the botanical basket is a container having partially or completely a porous or permeable outer surface and an open portion (for example, an open top), into which the solid component taken from the pre-soaking and liquid extraction step can be placed after it has been removed from the pre-soaking container. In various embodiments, the solid component can be placed within the botanical basket in a manner that maximizes the surface area that will be contacted by the steam supply. For example, in various embodiments, the solid component can be further pulverized into smaller solid pieces, or the amount of solid component can be controlled—e.g., less than the full amount pre-soaked can be put into the botanical batch in one run of the system, so that the solid pieces are spread out and their contact with the steam supply can be maximized.

In various embodiments, the botanical basket is configured to reside within the same housing as the boiler, or configured to reside in a housing separate from the boiler. That is, in certain embodiments, for example as shown in FIG. 2, the botanical basket 6 can be located within a separate housing 7 from the boiler 3; however, in various other embodiments, for example, as shown in FIG. 3, the botanical basket can be located within the same housing as the boiler, or can be configured to be contained partially or entirely within the boiler; and either in a housing or not in a housing. In an exemplary embodiment shown in FIG. 3, the botanical basket 6 holding the solid component 5 are in the same housing, and after the steam supply 4 flows through the botanical basket, enhanced vapor 10 is formed, which then exits the boiler and flows into the distillation column 11.

In certain embodiments, a boiler herein can be configured such that the botanical basket sits inside of it, but higher up in the boiler than the bottom surface of the boiler, such that any liquid sitting on the bottom surface of the boiler is, in various embodiments, not touching the bottom of the botanical basket when remaining in the liquid state. Thus, in certain embodiments, the botanical basket is contained within the boiler but configured to sit in a space in the proximity of the liquid component in the boiler, such that when the liquid component is boiled, the resultant vapor travels through the botanical basket and further contacts the solid component from the pre-soaking and liquid extraction step. This can result in an enhanced vapor stream 10 that is then directed to exit the boiler, or in certain embodiments, to exit the housing that contains the botanical basket, whether or not it is in the same housing as the boiler. In certain embodiments, the botanical basket can sit such that it is at least partially touching the liquid in the boiler.

In certain embodiments, the vapor stream that exits the botanical basket is an enhanced vapor stream. As used herein, “enhanced” means having more of any one or more of the desirable components than the steam supply that results from the boiling. This can be accomplished by the further contact of the steam supply 4 with the solid component 5 that is located in the botanical basket 6. This is an advantage of the methods and processes herein over what is currently known in the art.

In certain embodiments, the enhanced vapor stream 10 exits the top of the botanical basket (for example, as shown in FIG. 3), but the configuration of the botanical basket does not require that it exit from any particular side of the botanical basket, just that it passes through the biomass inside, maximizing the contact of the steam with the biomass so as to yield a vapor stream having an enhanced or optimal concentration of desired end product.

### Column and End Products

In certain embodiments, after passing through the botanical basket, the steam supply becomes an enhanced vapor stream 10, which can then be guided into a distillation column 11, as shown in FIG. 2 and FIG. 3. In various embodiments, the distillation column 11 is operated at a temperature sufficient to vaporize at least one desirable end product, such that the desirable end product is present in a distillate stream 13 that exits the top of the distillation column. From there, in certain embodiments, for example, as shown in FIG. 3, the distillate stream can enter a condenser 20 that is maintained at a lower temperature than the temperature of the distillate stream as it exits the distillation column, allowing for the creation of a condensate from at least a portion of the distillate stream. The remainder of the distillate stream can then be directed to a reflux drum 21 and then back into the distillation column for reheating. In various embodiments, reflux can be achieved after the botanical basket, column, or condenser. The desired end product can be sent to a receiver 18.

Similarly, as shown, for example, in FIG. 3, in certain embodiments, any retentate can exit the bottom of the distillation column in a retentate stream 12 that then enters a reboiler 14, out of which a reboiled vapor stream 17 can re-enter the distillation column. Any unvaporized material from the reboiler can exit as a bottoms stream 16 to a bottoms holding tank 15.

As can be seen in the embodiment shown in FIG. 2, in certain embodiments the desired end product or distillate stream 13 goes through a separator 22 to separate the desired end product (or multiple desired end products) 23. That is, in certain embodiments, the distillate stream 13 comprises at least one oil component and at least one aqueous component, and the separator 22 can separate the at least one oil component from the at least one aqueous component; for example, separating essential oil from floral waters. The oil component can comprise, for example, a plant oil (e.g., an essential oil, including but not limited to any natural oil such as rosehip oil, eucalyptus oil, moringa oil or the like). The aqueous component can comprise, for example, an aqueous extract from a plant, e.g., witch hazel, rice, cucumber, or the like.

In certain embodiments, any of the above-described steps can be conducted in more than one distillation column, as a way of speeding up the process and leading to greater efficiency of obtaining the final desired product or products.

### Vacuum

In certain embodiments, for example, as shown in FIG. 3, the any of the steps described herein are conducted under vacuum or pressurizing effect, applied by a pump 19. While the pump is shown in a certain configuration in FIG. 3, the location of the pump is not so limited, and can be placed anywhere in a method or process herein that serves to apply the vacuum or pressurizing effect on any portion of the method or process. In certain embodiments, any or all stages of the methods and processes herein can be subjected to a change in pressure—whether increased (i.e., pressurized) or decreased (i.e., subject to a vacuum). That is, in various embodiments, the pressure in any part of a method or process herein can be maintained by a vacuum or pressurizing device. In various embodiments, any change in pressure can be applied such that the pressure of any stage of a method or process herein can be maintained in a range of 200 mmHg (0.263 atm) to 760 mmHg (1 atm); or 200 mmHg (0.263 atm) to 1520 mmHg (2 atm); or 200 mmHg (0.263 atm) to 2280 mmHg (3 atm); or 200 mmHg (0.263 atm) to 3040 mmHg (1 atm). In various embodiments, the lower range can be near-absolute vacuum or pressurized to the limit of the equipment being used.

### Final Product

In certain embodiments, the final product exiting a process herein is highly desirable, in that it contains the desired botanical extract or material in a sufficiently concentrated amount that minimal (or no) further processing is required before obtaining the final commercial product.

Products produced as a result of the methods and processes herein have a wide variety of uses; among them, as ingredients in cosmetics and personal care products, home care products, or food or beverages.

Certain useful and desirable properties of the methods, processes and compositions herein can be illustrated in the following Examples.

### COMPARATIVE EXAMPLE

Initial (First Generation) Trials

Plant matter was pre-processed as necessary (i.e., fruits and vegetables were chopped into smaller pieces, dried herbs were sifted) and then mixed with water and added directly to a boiler (kettle). The water and plant mixture was brought to a boil (212° F.) and distillation continued for approximately 4 hours, until approximately 75% of the initial weight was collected in a receiver.

Visual observations of the hydrosol: dark coloration, off odors, low yield of plant actives, minimal effects when applied topically to subjects.

### Example 1

Second Generation Trials

Plant matter was pre-processed as necessary, as outlined above, and then mixed with deionized water and allowed to soak in a tank with the water for 2 to 18 hours. The solid material was removed from the liquid extract, and pressed. The liquid product of the pressing was mixed with the liquid extract and retained as the liquid component. The solid portion leftover after pressing (the solid component) was added to the botanical basket of the distillation setup. The kettle of the test still setup was charged with a specified weight of water and the total retained liquid extract (the liquid component) was added to this and brought to a boil (212° F.). Distillation continued for approximately 4 hours until approximately 75% of the initial weight (that is, the initial weight that was charged into the boiler (liquid portion of extraction+water) was collected in the receiver.

Visual observations of the hydrosol: medium coloration (above threshold of desirability as end product), off odors (milder than in the Comparative Example), significantly high yield of plant actives. Specifically, some hydrosols were evaluated using a tannin/lignin content test. Without a presoak of the witch hazel plant matter, no detectable tannins we present in the hydrosol. After incorporating the presoaking step, levels of 5+ ppm (up to 15 ppm) were achieved.

Topical sensory results: some tingling, tightening, soothing on the skin (depending on the plant material used).

### Example 2

Third Generation Trials

Plant matter was pre-processed as necessary, as outlined above, and then mixed with deionized water and allowed to soak in a tank with the water for 2 to 18 hours. The solid material was removed from the liquid extract, and pressed. The liquid product of the pressing was mixed with the liquid extract and retained as the liquid component. The solid portion leftover after pressing (the solid component) was added to the botanical basket of our distillation setup. The kettle of the test still setup was charged with a specified weight of water and the total retained liquid extract (the liquid component) was added to this.

The entire distillation setup was brought down to a vacuum of 200 mmHg. The kettle was brought to a boil (150° F.). This was advantageous, in that the lower pressure permitted boiling at a lower temperature, and therefore a cost and energy savings. The lower temperature also allowed for distillation without undesirable change of the natural state of plant matter (less breakdown, polymerization, deconformation, etc. of plant matter). Distillation continued for 2 hours until 95% of the initial weight was collected in the receiver.

Visual observations of the hydrosol: minimal coloration, mild and pleasant aromas, high yield of plant actives, similar to Example 1 above.

Based on Examples 1 and 2 above, the following was concluded:

A soaking step prior to distillation helped extract a higher level of plant actives.

A vacuum was applied to the whole system; this allowed vaporization (steam production) at a much lower temperature than would have been required in the absence of a vacuum. This decreased the overall amount of energy used, reduced the time a distillation run consumed, and enabled distillation at a temperature that did not cause excessive breakdown of the liquid extract in the kettle. As used herein, “breakdown” means undesirable change of the natural state of plant matter (e.g., polymerization, deconformation or any other undesirable alteration of plant matter).

The concept of using a botanical basket during distillation for the purposes of producing a hydrosol production was innovative and yielded desirable results in the end products of the process.

### Example 3

Exemplary Rosehip Hydrosol Procedure

Feed stock was prepared by weighing out 0.4 lbs. of whole rose hips and washing them to remove any excess dirt or contaminants. These were then contained in a food-grade mesh fabric, which was then fully submerged in 34 lbs. of RO/DI ultrapure water, and allowed to soak at ambient temperature (about 70° F.) for 18 hours, with occasional agitation to generate an extract. The solids were then removed from the liquid extract and transferred to the press. The liquid extract was added directly to the still.

The pressing step was performed in a Tico 40 Hydraulic Press. Pressure was applied to the rose hips gradually until no more liquid could be observed flowing to the stainless-steel collection basin. The liquid that was collected during pressing was transferred directly to the still. The pressed rose hips were transferred to the botanical basket column.

Distillation took place in a StillDragon 100 gallon Distillation System. An additional 367 lbs. of RO/DI ultrapure water was added to the extract liquid already in the still. Separately, the pressed rose hips were already loaded in the botanical basket column (located pre-condensation tube). This setup allows the steam generated in the kettle to percolate through the rose hips before condensation and collection.

To begin the distillation, a vacuum pump (Busch R5 Model RA0165) was used to reduce pressure inside the whole Distillation System. The system pressure was brought down to a vacuum of 200 mmHg. Heat was introduced to the kettle via its steam jacket and evaporation took place at 150 degrees F. Heat within the kettle was maintained at this level for the duration of distillation.

A stainless-steel conduit directed the distillate steam from the kettle through the botanical basket column to an 8″ Product Shotgun with Parrot Mount Condenser. A chiller was used to maintain the low temperature of the condenser. Condensate was captured in the collection tank (also kept under vacuum).

Distillation was halted after 385 lbs of distillate was collected. Distillation time was 120 minutes and was halted by returning the inside pressure of the system to atmospheric pressure and cutting off the heat supply. A yield of over 95% of the original weight was observed. The distillate was mixed under UV light then filtered through a sub micron filter to promote a microbial-free product. The rose hip hydrosol product was preserved with a plant based system and sealed in airtight non-reactive containers.

Notes Regarding Test Material and Parameter Variations:

A large variety of plant materials: herbs, vegetables, fruits, flowers, nuts, seeds, wood, bark, roots, twigs, etc. can be processed. All or part of a plant can be processed.

A combination of plants in a single run (rose hips and willow bark for example) can be distilled.

In certain embodiments, the following weights and ranges are specific to this unique test-still setup, and can be scaled virtually indefinitely. In various embodiments, the weight range of starting plant material is between 0.4 lbs and 6 lbs. In various embodiments, the amount of water this is soaked in can vary between 15 lbs and 45 lbs. In various embodiments, the amount of water that is used during distillation ranges from 400 lbs. to 800 lbs.

Pre-processing of the plant material can involve, in various embodiments: chopping, grinding, milling, fermenting or other similar bio and mechanical processing.

Temperature during the soaking step (pre-soaking or pre-distillation) is, in certain embodiments, carried out at room temperature, but can, in other embodiments, be heated (not above boiling) or chilled (down to near freezing) to obtain a more specific extraction. This can also be dependent on the liquid being used (various solvents have varying freeze/boil temperatures).

In various embodiments, there is no minimum or maximum time specified for the pre soaking step.

In various embodiments, plant matter can be added to the botanical basket without a soaking step.

In various embodiments, the time during the pressing step will vary depending on the material type. It will generally be on the order of minutes up to 2 or 3 hours.

The vacuum level for this test-still setup was in the range of 760 mmHg (zero vacuum/atmospheric pressure) and 200 mmHg (0.263 atm) of vacuum. In other embodiments, this range can be expanded to near-absolute vacuum or pressurized to the limit of the equipment being used. The temperature that evaporation occurs is dependent on this vacuum level.

Although the present technology has been described in relation to embodiments thereof, these embodiments and examples are merely exemplary and not intended to be limiting. Many other variations and modifications and other uses will become apparent to those skilled in the art. The present technology should, therefore, not be limited by the specific disclosure herein, and can be embodied in other forms not explicitly described here, without departing from the spirit thereof.

We claim:

1. A method for obtaining a natural ingredient from a biomass, the method comprising the steps of:
(a) pre-soaking a quantity of the biomass in a solvent, yielding a solid component and a liquid component;
(b) transferring the liquid component into a boiler, the boiler having a space through which a steam supply generated from the boiler moves;
(c) transferring the solid component into a botanical basket that is configured such that the steam supply generated from the boiler, when moving, passes through the botanical basket and contacts the solid component in the botanical basket, yielding an enhanced vapor stream that exits the botanical basket and enters a distillation column;
(d) passing the enhanced vapor stream through the distillation column, yielding a distillate stream,
(e) directing the distillate stream from the distillation column to a condenser maintained at a temperature lower than the temperature of the distillate stream as it exits the distillation column, allowing for the creation of a condensate from at least a portion of the distillate stream; and
(f) capturing the condensate in a collection tank;
wherein one or more of steps (a) through (f) is performed at a pressure of 200 mmHg (0.263 atm) to 760 mmHg (1 atm).

2. The method of claim 1, wherein the biomass comprises a plant.

3. The method of claim 1, wherein the solvent comprises water, alcohol, a plant oil, or glycerin.

4. The method of claim 1, wherein the pressure is maintained by a vacuum or pressurizing device.

5. The method of claim 1, wherein the botanical basket is configured to reside within the same housing as the boiler.

6. The method of claim 1, wherein the botanical basket is configured to reside in a housing separate from the boiler.

7. The method of claim 1, wherein the distillate stream comprises an oil component and an aqueous component.

8. The method of claim 7, further comprising a separator that separates the at least one oil component from the at least one aqueous component.
